(19) United States
(12) Patent Application Publication
(10) Pub. No.: US 2026/0069146 A1
(43) Pub. Date: Mar. 12, 2026

(54) POSITION-DEPENDENT STIMULATION MONITORING OF THE HEART

(71) Applicant: Siemens Healthineers AG, Forchheim (DE)

(72) Inventors: Manuela Rösler, Erlangen (DE); Daniel Niederlöhner, Erlangen (DE)

(21) Appl. No.: 19/325,468

(22) Filed: Sep. 10, 2025

(30) Foreign Application Priority Data
Sep. 12, 2024 (DE) 10 2024 208 679.7

(51) Int. Cl. A61B 5/00; A61B 5/055

(52) U.S. Cl. CPC A61B 5/0037; A61B 5/0044; A61B 5/055

(57) ABSTRACT

A method for ascertaining at least one monitoring parameter for monitoring cardiac stimulation during a magnetic resonance measurement of a patient, a magnetic resonance apparatus, and a computer program product are provided. According to the method, the patient is positioned in the magnetic resonance apparatus. The cardiac position of the patient relative to the magnetic resonance apparatus is ascertained. The at least one monitoring parameter is ascertained based on a monitoring model as a function of the cardiac position of the patient.

This application claims the benefit of German Patent Application No. DE 10 2024 208 679.7, filed on Sep. 12, 2024, which is hereby incorporated by reference in its entirety.

### BACKGROUND

The present embodiments relate to ascertaining at least one monitoring parameter for monitoring cardiac stimulation during a magnetic resonance measurement of a patient.

In medical engineering, imaging is characterized by high soft tissue contrast by magnetic resonance (MR), also referred to as magnetic resonance tomography (MRT) or magnetic resonance imaging (MRI). A magnetic resonance measurement is performed by a magnetic resonance apparatus that has a magnet unit. The magnet unit may include a main magnet for generating a main magnetic field and a gradient coil unit for generating a gradient magnetic field in an examination region of the magnetic resonance apparatus. The examination region may be located in a magnet bore (e.g., a bore) of the magnet unit, in which a patient is positioned during the magnetic resonance measurement. To generate magnetic resonance signals during a magnetic resonance measurement, radiofrequency (RF) transmit pulses are emitted into the examination region according to a magnetic resonance sequence.

The gradient magnetic fields generated during a magnetic resonance measurement may lead to nerve stimulation of the patient (e.g., of his heart). In order to exclude any danger to the patient due to excessively intense nerve stimulation, in accordance with the standard IEC 60601-2-33, the generated gradient magnetic fields may be a cylindrical monitoring volume. The monitoring volume has a cylinder axis that corresponds to the patient axis, a radius of 0.20 m, and a length equal to the length of the gradient coil unit. The monitoring, for which the SAFE model described in Hebrank FX, Gebhardt M. SAFE model—A new method for predicting peripheral nerve stimulation in MRI; Proceedings of the 8th Annual Meeting of ISMRM; Denver. 2000; p. 2007 is used for example, provides that predetermined limit values, such as a maximum change over time of the magnetic field dB/dt for example, are not exceeded in the overall monitoring volume. This may lead to the performance capability of the magnetic resonance measurement being considerably limited.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, performance limitations of magnetic resonance measurements that are caused by monitoring cardiac stimulation may be minimized without accepting an increased risk to the patient.

A method (e.g., computed-implemented method) is proposed for ascertaining at least one monitoring parameter for monitoring cardiac stimulation (e.g., cardiac nerve stimulation) during a magnetic resonance measurement of a patient. In this context, the patient is positioned in the magnetic resonance apparatus. The cardiac position (e.g., position of the heart) of the patient relative to the magnetic resonance apparatus is ascertained. The at least one monitoring parameter is ascertained based on a monitoring model as a function of the cardiac position.

The ascertaining of a cardiac position of the patient relative to the magnetic resonance apparatus and/or the ascertaining of the at least one monitoring parameter based on a monitoring model as a function of the cardiac position of the patient may be performed, for example, by an ascertaining unit. The ascertaining unit may include, for example, one or more (e.g., programmable) processors and/or one or more storage modules. For example, the monitoring model may be stored in a memory and loaded from the memory in order to carry out the method.

The monitoring model may include the SAFE model described in Hebrank FX, Gebhardt M. SAFE model—A new method for predicting peripheral nerve stimulation in MRI; Proceedings of the 8th Annual Meeting of ISMRM; Denver. 2000; p. 2007, for example. The at least one monitoring parameter is, for example, a parameter of the SAFE model. One such monitoring parameter may be a scaling factor, for example, that describes the maximum intensity of a gradient magnetic field of a gradient coil in a particular volume.

The positioning of the patient may be performed and/or supported, for example, by a member of operating personnel of the magnetic resonance apparatus. In one embodiment, the patient is positioned in a magnet bore of the magnetic resonance apparatus, so that at least one part of the patient (e.g., the heart of the patient) is located in the magnet bore. For example, the patient is laid on a patient table, which is then moved (e.g., together with the patient) into a magnet bore of the magnetic resonance apparatus.

In one embodiment, the patient assumes an examination position due to the positioning in the magnetic resonance apparatus. In one embodiment, the patient is also located in this examination position even during a subsequent magnetic resonance measurement.

For example, the cardiac position may be ascertained relative to a reference position (e.g., reference location) of the magnetic resonance apparatus. For example, the cardiac position may be ascertained relative to a gradient coil unit of the magnetic resonance apparatus.

The at least one monitoring parameter may include, for example, a monitoring volume and/or a monitoring value that is derived therefrom and/or dependent thereupon. One possible monitoring value that is derived therefrom and/or dependent thereupon may be a scaling factor, for example, that describes the maximum intensity of a gradient magnetic field of the gradient coil unit in the monitoring volume. In one embodiment, smaller scaling factors are ascertained for smaller monitoring volumes that have a smaller distance from the isocenter. In one embodiment, these smaller scaling factors are used as monitoring parameters for an adapted parameterization of the monitoring of the cardiac stimulation, which, for example, may be configured as a monitoring model using the SAFE model.

In one embodiment, by taking into consideration the cardiac position when ascertaining the at least one monitoring parameter, it is possible to avoid unnecessary safety margins that limit the performance capability of the magnetic resonance measurements. In one embodiment, the monitoring volume may be made smaller compared to the prior art, by the monitoring volume being adapted to the actual position of the heart of the patient. In one embodiment, it is possible to avoid a blanket definition of the monitoring volume that is not specific to the positioning.

In one embodiment, a magnetic resonance measurement is performed, where the magnetic resonance measurement is monitored based on the at least one monitoring parameter. The monitoring may take place with a cardiac stimulation monitor, for example, which is parameterized according to the at least one monitoring parameter.

In one embodiment, the monitoring of the cardiac stimulation during the magnetic resonance measurement according to the at least one monitoring parameter is suitable for restricting a stimulation of the heart of the patient. In one embodiment, the patient safety is increased due to the monitoring of the cardiac stimulation during the magnetic resonance measurement.

In one embodiment, the performance capability of a magnetic resonance measurement monitored in such a manner may be increased. In one embodiment, the magnetic resonance measurement may be performed more quickly, and/or safety-related measurement terminations may be avoided. In one embodiment, magnetic resonance sequences that have a higher level of gradient activity (e.g., cause a greater intensity of a gradient magnetic field generated according to the magnetic resonance sequence) may be used, and/or greater rates of change (e.g., slew rates) of the gradient magnetic field may be permitted, which, for example, may lead to shorter rise times when a constant gradient intensity is to be achieved.

One possible embodiment of the method provides that, while the magnetic resonance measurement is being performed, the cardiac position of the patient relative to the magnetic resonance apparatus is ascertained repeatedly and the at least one monitoring parameter is ascertained (e.g., adapted) based on the monitoring model as a function of the repeatedly ascertained cardiac position of the patient.

In one embodiment, the (re-)ascertaining of the cardiac position and possibly a change in the at least one monitoring parameter takes place on a continuous and/or ongoing basis while the magnetic resonance measurement is being performed. In one embodiment, the monitoring of cardiac stimulation may thus be adapted to a possible change in the cardiac position. A change in the cardiac position may be caused, for example, by a movement of the patient and/or of a patient table carrying the patient.

One further possible embodiment of the method provides that the magnetic resonance apparatus includes a gradient coil unit. The monitoring model includes a location-dependent intensity of a gradient magnetic field that may be generated by the gradient coil unit (e.g., gradient intensity). For example, the location-dependent intensity of a gradient magnetic field that may be generated by the gradient coil unit is the location-dependent intensity of the gradient magnetic field that is generated when a particular current flows through at least one gradient coil of the gradient coil unit.

For example, the gradient intensity may be the amplitude of the gradient magnetic field. For example, the gradient intensity may be indicated in the unit of measurement mT/m.

The gradient coil unit may include three gradient coils for example that, for example, may generate gradient magnetic fields in different directions (e.g., directions that are perpendicular to one another). A first of the three gradient coils (e.g., x-gradient coil), for example, may generate a gradient magnetic field along a first direction (e.g., an x-direction). A second of the three gradient coils (e.g., y-gradient coil), for example, may generate a gradient magnetic field along a second direction (e.g., a y-direction). A third of the three gradient coils (e.g., z-gradient coil), for example, may generate a gradient magnetic field along a third direction (e.g., a z-direction). For example, the location-dependent intensity of the gradient magnetic field that may be generated by the z-gradient coil is the generable intensity of the gradient magnetic field as a function of a position in the z-direction. This also applies to the x-direction and y-direction in a similar manner.

For example, a gradient coil may include two partial gradient coils that may be operated in pairs with the same current intensity but opposing polarity. In one embodiment, the one partial gradient coil increases the magnetic field by an absolute value, while the opposing partial gradient coil reduces the magnetic field by the same absolute value. The magnetic field may be altered overall as a result.

In one embodiment (e.g., in cardiac magnetic resonance tomography), the heart is positioned in the isocenter or in the vicinity of the isocenter of the magnetic resonance apparatus. The main magnetic field of the magnetic resonance apparatus may have a particularly high level of homogeneity in the isocenter.

In one embodiment, the center point of the gradient coil unit is located in the isocenter. In one embodiment, the gradient coils of the gradient coil unit are arranged symmetrically in relation to the isocenter. In one embodiment, the center points of the gradient coils of the gradient coil unit may be located in the isocenter in each case.

However, a magnetic resonance apparatus that has an asymmetrical arrangement of the gradient coils may also be provided.

Typically, the intensity of the gradient magnetic field (e.g., the gradient magnetic field that may be generated by the z-gradient coil) has a zero-crossing in the isocenter, and the technically achievable field intensity is lower than in the outer region of the gradient coil. The location-dependent intensity of the gradient magnetic field that may be generated by the gradient coil unit is thus typically relatively low in the isocenter.

As a result, the requirements for the monitoring of a cardiac stimulation may be lowered in a particularly effective manner if, through the ascertaining of the cardiac position, it is identified that the heart of the patient is located in the isocenter or in the vicinity of the isocenter.

One further possible embodiment of the method provides that, in order to ascertain the at least one monitoring parameter, based on the cardiac position, a spatial region (e.g., monitoring region) is ascertained in which a predefined stimulation is not to be exceeded. The at least one monitoring parameter is ascertained as a function of the ascertained spatial region.

The spatial region may be, for example, a volume (e.g., a spatial volume). The predefined stimulation may be, for example, a nerve stimulation. The predefined stimulation may be, for example, a stimulation of the heart of the patient. The prescribed stimulation may be defined by a standard, for example.

In one embodiment, the monitoring region includes the cardiac position of the patient. In one embodiment, the monitoring region is smaller than the monitoring volume to be monitored in accordance with the standard IEC 60601-2-33, which was described in the foregoing. In one embodiment, a less precise ascertaining of the cardiac position is also already sufficient, in order to make the monitoring volume considerably smaller compared to what is specified in the standard IEC 60601-2-33. The performance limitations may only be applied to the monitoring volume that has been made smaller. This makes it possible to choose the requirements for the monitoring or the at least one monitoring parameter such that the monitoring is less restrictive.

In one embodiment, at least one control value for controlling the magnetic resonance apparatus is ascertained. The monitoring of the cardiac stimulation includes a monitoring of the at least one control value. For example, the at least one control value may relate to a current flow through the gradient coil unit (e.g., through gradient coil unit).

In one embodiment, due to the monitoring of the at least one control value, it may be provided that a predefined stimulation (e.g., in the monitoring region) is not exceeded.

One possible embodiment of the method provides that the ascertaining of a cardiac position takes place based on a magnetic resonance image of the patient. In one embodiment, magnetic resonance images are particularly well suited for mapping internal organs of the patient, and thus also the heart. In one embodiment, magnetic resonance images are particularly well suited for mapping soft tissue, and thus also the heart. This also makes it possible to ascertain the position of the heart in a reliable manner based on a magnetic resonance image.

The magnetic resonance signals required for reconstruction of the magnetic resonance image may be captured by the magnetic resonance apparatus, once the patient has been positioned in the magnetic resonance apparatus.

In one embodiment, the magnetic resonance image is recorded before a main measurement of the magnetic resonance measurement, where at least one measurement parameter of the main measurement is set based on the magnetic resonance image. The setting of the at least one measurement parameter may take place, for example, via a member of operating personnel of the magnetic resonance apparatus. For example, the member of operating personnel is shown the magnetic resonance image, after which he sets the at least one measurement parameter of the main measurement based on the magnetic resonance image.

A magnetic resonance examination may include the performance of multiple magnetic resonance measurements (e.g., a pre-measurement and a subsequent main measurement). The magnetic resonance signals necessary for the magnetic resonance image, based on which the cardiac position is determined, may be captured in such a pre-measurement using the magnetic resonance apparatus. A pre-measurement of this kind may be a localization measurement (e.g., localizer measurement), for example, based on which a slice to be measured in the main measurement is set as measurement parameter of the main measurement, for example. For example, a slice orientation of the main measurement is planned based on the data of the localization measurement. For example, the planning may take place on a manual or automated basis.

In one embodiment, the pre-measurement is performed anyway as part of the magnetic resonance examination, such that the recording of the magnetic resonance image, based on which the cardiac position is ascertained, may be performed in a time-efficient manner.

One further possible embodiment of the method provides that the ascertaining of the cardiac position takes place based on a camera image of the patient. The camera image may be recorded by a camera. For example, the camera may be a 3D camera configured to capture (e.g., generate) three-dimensional images. In one embodiment, the camera is an optical camera. In one embodiment, the camera is configured to capture visible light and/or UV radiation and/or infrared radiation. In one embodiment, the camera is configured to capture electromagnetic wavelengths in a wavelength range between 400 and 780 nm.

The camera may be arranged inside or outside the magnet bore. For example, the camera is arranged on an inside wall of the magnet bore. In one embodiment, the camera has a field of view that includes the isocenter of the magnetic resonance apparatus.

In one embodiment, the external form of the patient may be captured by a camera image. Based on the external form of the patient, the cardiac position may be ascertained with sufficient accuracy. In one embodiment, in order to ascertain the cardiac position based on a camera image, a body model that describes the position of the heart in the body is used. In one embodiment, a general, non-patient-specific body model may be used, which still enables a sufficiently accurate ascertaining of the cardiac position.

One further possible embodiment of the method provides that the ascertaining of the cardiac position takes place based on a pilot tone signal. The pilot tone signal may be recorded by a radiofrequency antenna unit, for example. For example, the radiofrequency antenna unit may include one or more local coils. For example, the radiofrequency antenna unit may also be configured to record magnetic resonance signals. For example, a pilot tone transmitter generates a transmit signal. This transmit signal interacts with the patient, and the pilot tone signal resulting therefrom is then recorded. In one embodiment, the transmit signal has a frequency that is not equal to a Lamor frequency of the magnetic resonance apparatus. In one embodiment, the radiofrequency antenna unit has multiple receive antennas for receiving the pilot tone signal.

The beating heart of the patient may cause location-dependent magnetic field changes in the order of magnitude of nanotesla. This effect may be used to trigger cardiac measurements, for example, by the pilot tone signal being modulated by these effects. For example, if the pilot tone signal is recorded with multiple receive antennas (e.g., that are part of a local coil that is arranged directly on the patient), it is possible to infer the cardiac position based on the pilot tone signal. In this context, the position of the local coil relative to the magnetic resonance apparatus may be known, for example, with the aid of a Hall effect sensor that is arranged on or in the local coil.

Further, a magnetic resonance apparatus that is configured to carry out a method described in the foregoing for ascertaining at least one monitoring parameter for monitoring cardiac stimulation during a magnetic resonance measurement of a patient is provided.

The advantages of the magnetic resonance apparatus of the present embodiments essentially correspond to the advantages of the method of the present embodiments for ascertaining at least one monitoring parameter for monitoring cardiac stimulation during a magnetic resonance measurement of a patient that are explained above in detail. Features, advantages, or alternative embodiments mentioned herein may also be transferred similarly to the other subject matter and vice versa.

In addition to the components that a magnetic resonance apparatus may include, the magnetic resonance apparatus, for example, may include a camera (e.g., a 3D camera).

For example, the magnetic resonance apparatus may include a system control unit that is configured to ascertain a cardiac position of the patient relative to the magnetic resonance apparatus and the at least one monitoring parameter based on a monitoring model as a function of the cardiac position. For example, the system control unit may include an ascertaining unit described above.

Further, a computer program product is provided that includes a program and may be loaded directly into a memory of a programmable system control unit of a magnetic resonance apparatus and has program code means (e.g., libraries and auxiliary functions) in order to carry out a method provided in the foregoing for ascertaining at least one monitoring parameter for monitoring cardiac stimulation during a magnetic resonance measurement of a patient when the computer program product is executed in the system control unit of the magnetic resonance apparatus. In this context, the computer program product may include a piece of software with a source code that is still to be compiled and linked or only has to be interpreted, or an executable software code that only has to be loaded into the system control unit for execution.

Via the computer program product, the method of the present embodiments may be carried out in a rapid, identically repeatable and robust manner. In one embodiment, the computer program product is configured such that the computer program product may carry out the method steps of the present embodiments by the system control unit. In this context, the system control unit in each case has the prerequisites, such as a corresponding working memory, a corresponding graphics card, or a corresponding logic unit, for example, such that the respective method steps may be carried out efficiently.

The computer program product is saved on a computer-readable medium (e.g., a non-transitory computer-readable storage medium) or stored on a network or server, for example, from where it may be loaded into the processor of a local system control unit.

The processor of the local system control unit may be configured as directly connected to the magnetic resonance apparatus or as part of the magnetic resonance apparatus. Further, control information of the computer program product may be saved on an electronically readable data carrier. The control information of the electronically readable data carrier may be configured such that the control information performs a method of the present embodiments when the data carrier is used in a system control unit of a magnetic resonance apparatus.

Examples of electronically readable data carriers are a DVD, a magnetic tape, or a USB stick, on which electronically readable control information (e.g., software) is saved. If this control information is read from the data carrier and saved into a system control unit of the magnetic resonance apparatus, it is possible to perform all embodiments of the method described above.

Further advantages, features, and details of the present embodiments are disclosed in the example embodiments described in the following, as well as based on the drawings. Parts that correspond to one another are provided with the same reference characters in all figures.

### BRIEF DESCRIPTION OF THE DRAWINGS

FIG. 1 shows a magnetic resonance apparatus in a schematic representation;

FIG. 2 shows a diagram of a method for ascertaining at least one monitoring parameter for monitoring cardiac stimulation during a magnetic resonance measurement of a patient;

FIG. 3 shows a diagram of an expanded method for ascertaining at least one monitoring parameter for monitoring cardiac stimulation during a magnetic resonance measurement of a patient; and

FIG. 4 shows a representation of a location-dependent intensity of a gradient magnetic field that can be generated by the gradient coil unit.

### DETAILED DESCRIPTION

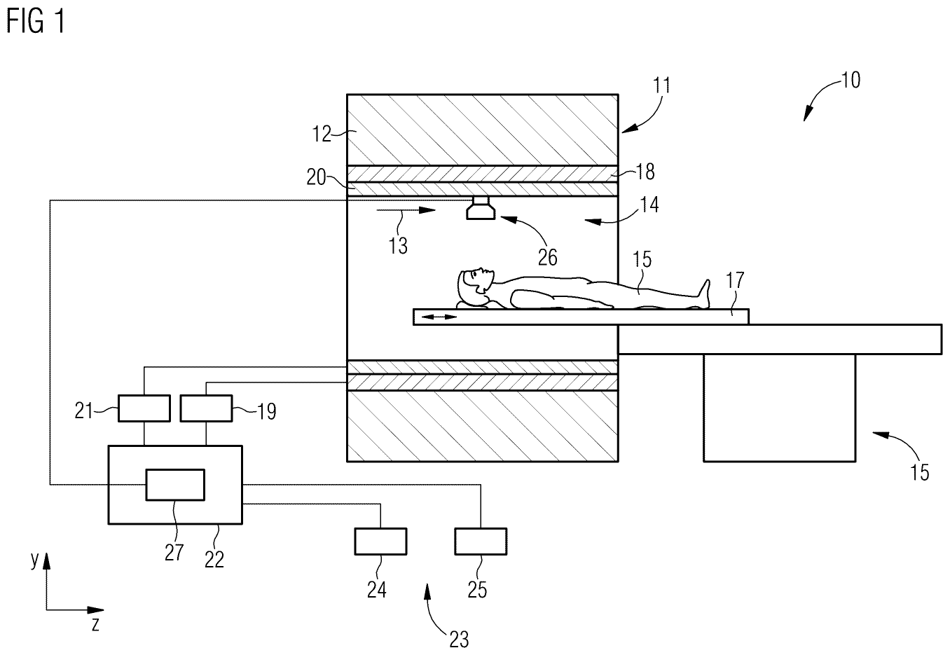

FIG. 1 is a schematic representation of one embodiment of a magnetic resonance apparatus 10. The magnetic resonance apparatus 10 includes a magnet unit 11 that has a main magnet 12 for generating an intense and, for example, temporally constant main magnetic field 13. The magnetic resonance apparatus 10 also includes a magnet bore 14 for accommodating a patient 15. In this example, the magnet bore 14 is configured as cylindrical and surrounded cylindrically by the magnet unit 11 in a circumferential direction. However, other shapes of magnet (e.g., an open magnet) may also be provided. Using a patient positioning apparatus 16 of the magnetic resonance apparatus 10, the patient 15 may be inserted into the magnet bore 14 and thus positioned in the magnetic resonance apparatus 10. The patient positioning apparatus 16 has a patient table 17 that is configured such that the patient table 17 may move inside the magnet bore 14.

The magnet unit 11 also has a gradient coil unit 18 for generating magnetic field gradients that are used for spatial encoding during an imaging. The gradient coil unit 18 may include multiple gradient coils that are not shown in detail here. For example, each of the multiple gradient coils may generate a gradient magnetic field in a spatial direction (e.g., a z-gradient coil in a z-direction and a y-gradient coil in a y-direction). The gradient coil unit 18 is controlled by a gradient control unit 19 of the magnetic resonance apparatus 10.

The magnet unit 11 also includes a radiofrequency antenna unit 20 that in the present example embodiment is embodied as a body coil permanently integrated into the magnetic resonance apparatus 10. The radiofrequency antenna unit 20 is controlled by a radiofrequency antenna control unit 21 of the magnetic resonance apparatus 10 and emits radiofrequency magnetic resonance sequences into an examination space that is substantially formed by the region of the magnet bore 14 of the magnetic resonance apparatus 10. As a result, an excitation of atomic nuclei ensues for the main magnetic field 13 generated by the main magnet 12. Magnetic resonance signals are generated due to relaxation of the excited atomic nuclei. The radiofrequency antenna unit 20 is configured to receive the magnetic resonance signals.

To control the main magnet 12, the gradient control unit 19 and to control radiofrequency antenna control unit 21, the magnetic resonance apparatus 10 has a system control unit 22. The system control unit 22 controls the magnetic resonance apparatus 10 centrally (e.g., the performance of a predetermined imaging magnetic resonance sequence). The system control unit 22 also includes an evaluation unit (not shown in further detail) for evaluating the magnetic resonance signals that are captured during the magnetic resonance examination. Further, the magnetic resonance apparatus 10 includes a user interface 23 that is connected to the system control unit 22. Control information, such as, for example, imaging parameters and reconstructed magnetic resonance mappings, may be displayed on a display unit 24 (e.g., on at least one monitor) of the user interface 23 for a member of medical operating personnel. Further, the user interface 23 has an input unit 25 by which information and/or parameters may be input by the member of medical operating personnel during a measurement procedure.

The magnetic resonance apparatus 10 further includes a camera 26 that, for example, is arranged on an inside wall of the magnet bore 14. However, the camera may also be arranged at another location (e.g., outside the magnet bore 14). The camera 26 is configured to capture camera images (e.g., also images of the patient 15) when the patient is located in the magnet bore 14.

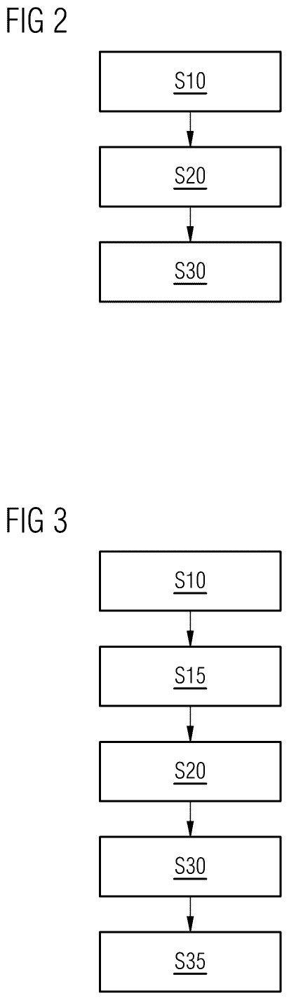

The camera images may be provided to an ascertaining unit 27 that, for example, is part of the system control unit 22. With the aid of the ascertaining unit, it is possible to perform the method shown in FIG. 2 for ascertaining at least one monitoring parameter for monitoring cardiac stimulation during a magnetic resonance measurement of a patient 15 (e.g., S20 and/or S30).

In this context, in S10, the patient 15 is positioned in the magnetic resonance apparatus 10 (e.g., inside the magnet bore 14). In S20, the cardiac position of the patient 15 positioned in the magnetic resonance apparatus 10 relative to the magnetic resonance apparatus 10 is ascertained. In S30, at least one monitoring parameter is ascertained based on a monitoring model as a function of the cardiac position of the patient 15. In one embodiment, in this context, any inaccuracies (e.g., measurement inaccuracies) when ascertaining the cardiac position are taken into consideration in S20.

In one embodiment, monitoring of the cardiac stimulation already takes place before the ascertaining of the at least one monitoring parameter as a function of the cardiac position in S30, in which conventional monitoring parameters (e.g., in accordance with the standard IEC 60601-2-33) are applied.

In FIG. 3, an expanded method is shown, where the following deals with aspects that are additional compared to FIG. 2. In this context, in S15, an image of the patient 15 is recorded, once the patient has been positioned in the magnetic resonance apparatus 10. The ascertaining of the cardiac position in S20 may take place based on the image recorded in S15. The image may be a camera image, for example, that is recorded with the camera 26.

Further, in one embodiment, in S15, a magnetic resonance image is recorded before a main measurement of the magnetic resonance measurement. The ascertaining of the cardiac position may then take place based on the magnetic resonance image of the patient 15 recorded in S15.

The main measurement of the magnetic resonance measurement is performed in S35, where the main measurement is monitored based on the at least one monitoring parameter. In one embodiment, based on the image recorded in S15 (e.g., a magnetic resonance image recorded in S15), at least one measurement parameter of the main measurement is also set based on the image.

In one embodiment, a recording of a current image S15 in each case, an ascertaining of a current cardiac position in S20, and an updating of the at least one monitoring parameter in S30 during the magnetic resonance measurement are repeatedly performed in S35 in order to be able to adapt the monitoring with respect to possible changes in the cardiac position of the patient 15. For example, in one embodiment, the cardiac position relative to the magnetic resonance apparatus changes due to a movement of the patient 15 himself and/or due to a movement of the patient table 17.

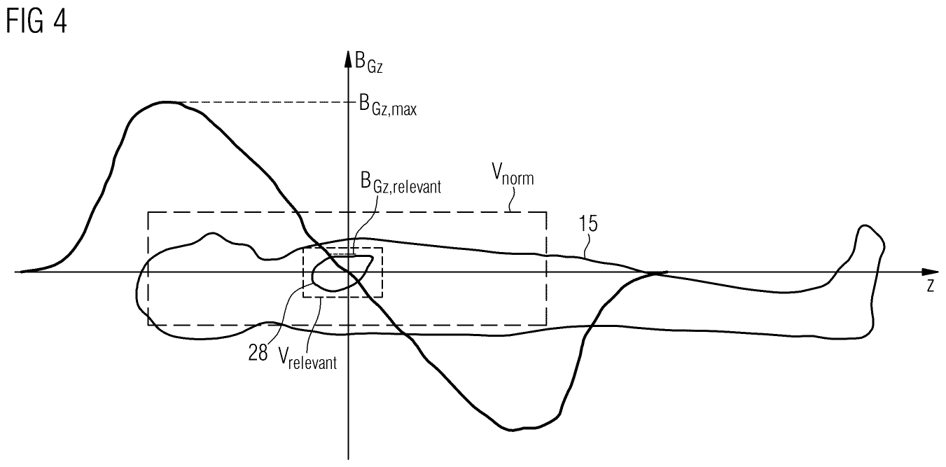

Further aspects are explained on the basis of FIG. 4. During a magnetic resonance examination of the heart, the heart 28 of the patient 15 may be positioned in the isocenter of the magnet unit 11. The position of the isocenter along the z-direction is characterized by the axis B_{Gz} here. In this context, B_{Gz} indicates the intensity of a gradient magnetic field in the z-direction that may be generated by the gradient coil unit 18 (e.g., a z-gradient coil of the gradient coil unit 18). At different z-positions, the intensity of the gradient magnetic field B_{Gz} has different values (e.g., this involves a location-dependent intensity). In one embodiment, this information is a constituent part of the monitoring model, with which the at least one monitoring parameter is ascertained in S30.

The example shown in FIG. 4 relates to the z-direction or the z-gradient coil. However, a corresponding generalization in relation to the x-direction or the x-gradient coil and the y-direction or y-gradient coil is also possible.

Conventionally (e.g., in accordance with the standard ICE 60601-2-33), a monitoring volume Vₙₒᵣₘ is predefined, which does not take into consideration the actual position of the heart 28. In this comparatively large monitoring volume Vₙₒᵣₘ, the intensity of the gradient magnetic field B_{Gz} may have comparatively high values. Accordingly, the monitoring parameters for monitoring the cardiac stimulation also have to be chosen in a correspondingly restrictive manner.

The gradient magnetic field B_{Gz} has a zero-crossing in the isocenter and increases toward the outside again, until its maximum value B_{Gz,max}, before it drops again. If the heart 28 of the patient 15 is located close to the isocenter, then the heart is also exposed to a comparatively small gradient magnetic field B_{Gz}. Consequently, the risk of excessively intense cardiac stimulation due to the gradient magnetic field is also low. Accordingly, the at least one monitoring parameter in S30 is set such that the corresponding monitoring of the cardiac stimulation is less restrictive. It is thus possible to avoid an unnecessary, excessive limitation of the gradient activity during a magnetic resonance measurement (e.g., the main measurement in S35).

For example, based on the cardiac position ascertained in S20, it is possible to ascertain a spatial region Vrelevant in which a predefined stimulation is not to be exceeded. In one embodiment, in S30, the at least one monitoring parameter is ascertained as a function of the ascertained spatial region Vrelevant.

For example, in this context, the maximum intensity of the gradient magnetic field B_{Gz} in the spatial region Vrelevant is ascertained, and this is indicated as B_{Gz,relevant} in FIG. 4. In one embodiment, the at least one monitoring parameter is ascertained under the boundary condition that the gradient magnetic field B_{Gz} does not exceed the value B_{Gz,relevant}. This permits less restrictive monitoring if, without considering the current cardiac position, a greater value for the maximum intensity of the gradient magnetic field would have to be assumed in the worst case. For example, B_{Gz,relevant} is an input value for the monitoring model that is applied in S30.

It is again noted that the method described above in detail in the foregoing and the magnetic resonance apparatus shown are merely example embodiments that may be modified by the person skilled in the art in a wide variety of ways without departing from the scope of the invention. Further, the use of the indefinite article “a” or “an” does not preclude the possibility that the relevant features may also be present plurally. Similarly, the expression “unit” does not preclude the relevant components consisting of a plurality of interacting subcomponents that may also be spatially distributed, if appropriate. Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for ascertaining at least one monitoring parameter for monitoring cardiac stimulation during a magnetic resonance measurement of a patient, the method comprising:
positioning the patient in a magnetic resonance apparatus;
ascertaining a cardiac position of the patient relative to the magnetic resonance apparatus; and
ascertaining the at least one monitoring parameter based on a monitoring model as a function of the cardiac position of the patient.

2. The method of claim 1, further comprising:
performing the magnetic resonance measurement,
wherein the magnetic resonance measurement is monitored based on the at least one monitoring parameter.

3. The method of claim 2, wherein, while the magnetic resonance measurement is being performed, the cardiac position of the patient relative to the magnetic resonance apparatus is ascertained repeatedly, and the at least one monitoring parameter is ascertained based on the monitoring model as a function of the repeatedly ascertained cardiac position of the patient.

4. The method of claim 1, wherein the magnetic resonance apparatus comprises a gradient coil unit,
wherein the monitoring model comprises a location-dependent intensity of a gradient magnetic field that is generatable by the gradient coil unit.

5. The method of claim 1, wherein the ascertaining of the at least one monitoring parameter based on the monitoring model as a function of the cardiac position of the patient comprises:
ascertaining a spatial region, in which a predefined stimulation is not to be exceeded, based on the cardiac position; and
ascertaining the at least one monitoring parameter as a function of the ascertained spatial region.

6. The method of claim 1, wherein ascertaining the cardiac position comprises ascertaining the cardiac position of the patient based on a magnetic resonance image of the patient.

7. The method of claim 6, wherein the magnetic resonance image is recorded before a main measurement of the magnetic resonance measurement, and
wherein at least one measurement parameter of the main measurement is set based on the magnetic resonance image.

8. The method of claim 1, wherein ascertaining the cardiac position comprises ascertaining the cardiac position of the patient based on a camera image of the patient.

9. The method of claim 1, wherein ascertaining the cardiac position comprises ascertaining the cardiac position of the patient based on a pilot tone signal.

10. A magnetic resonance comprising:
a processor configured to ascertain at least one monitoring parameter for monitoring cardiac stimulation during a magnetic resonance measurement of a patient, the processor being configured to ascertain the at least one monitoring parameter comprising the processor being configured to:
position the patient in a magnetic resonance apparatus;
ascertain a cardiac position of the patient relative to the magnetic resonance apparatus; and
ascertain the at least one monitoring parameter based on a monitoring model as a function of the cardiac position of the patient.

11. In a non-transitory computer-readable storage medium that stores instructions executable by one or more processors to ascertain at least one monitoring parameter for monitoring cardiac stimulation during a magnetic resonance measurement of a patient, the instructions comprising:
positioning the patient in a magnetic resonance apparatus;
ascertaining a cardiac position of the patient relative to the magnetic resonance apparatus; and
ascertaining the at least one monitoring parameter based on a monitoring model as a function of the cardiac position of the patient.

12. The non-transitory computer-readable storage medium of claim 11, wherein the instructions further comprise:
performing the magnetic resonance measurement,
wherein the magnetic resonance measurement is monitored based on the at least one monitoring parameter.

13. The non-transitory computer-readable storage medium of claim 12, wherein, while the magnetic resonance measurement is being performed, the cardiac position of the patient relative to the magnetic resonance apparatus is ascertained repeatedly, and the at least one monitoring parameter is ascertained based on the monitoring model as a function of the repeatedly ascertained cardiac position of the patient.

14. The non-transitory computer-readable storage medium of claim 11, wherein the magnetic resonance apparatus comprises a gradient coil unit,
wherein the monitoring model comprises a location-dependent intensity of a gradient magnetic field that is generatable by the gradient coil unit.

15. The non-transitory computer-readable storage medium of claim 11, wherein the ascertaining of the at least one monitoring parameter based on the monitoring model as a function of the cardiac position of the patient comprises:
ascertaining a spatial region, in which a predefined stimulation is not to be exceeded, based on the cardiac position; and
ascertaining the at least one monitoring parameter as a function of the ascertained spatial region.

16. The non-transitory computer-readable storage medium of claim 11, wherein ascertaining the cardiac position comprises ascertaining the cardiac position of the patient based on a magnetic resonance image of the patient.

17. The non-transitory computer-readable storage medium of claim 16, wherein the magnetic resonance image is recorded before a main measurement of the magnetic resonance measurement, and
wherein at least one measurement parameter of the main measurement is set based on the magnetic resonance image.

18. The non-transitory computer-readable storage medium of claim 11, wherein ascertaining the cardiac position comprises ascertaining the cardiac position of the patient based on a camera image of the patient.

19. The non-transitory computer-readable storage medium of claim 11, wherein ascertaining the cardiac position comprises ascertaining the cardiac position of the patient based on a pilot tone signal.
